(19) United States
(12) Patent Application Publication
(10) Pub. No.: US 2006/0034952 A1
(43) Pub. Date: Feb. 16, 2006

(54) Agent for inhibiting the growth of mammalian hair

(76) Inventor: Mrinmayee Bhushan Kondhalkar, Pune (IN)

Correspondence Address: NOTARO AND MICHALOS, 100 DUTCH HILL ROAD, SUITE 110, ORANGEBURG, NY, 10962-2100, US

(21) Appl. No.: 10/988,804

(22) Filed: Nov. 15, 2004

(30) Foreign Application Priority Data
Aug. 13, 2004 (IN) 873/MUM/04

(51) Int. Cl. A61K 36/47; A61K 8/64

(52) U.S. Cl. 424/731; 424/70.14

(57) ABSTRACT

The present invention comprises of a composition for post-hair-removal topical application for inhibiting mammalian hair growth & application thereof. The present invention also embraces a method of inhibiting mammalian hair growth by applying an effective amount of the composition to the skin. The composition comprises of a hair growth-inhibiting agent and a dermatologically acceptable vehicle.

### PRIOR ART

Generally normal body hair growth is tolerated. But if there is excessive unwanted hair growth it affects the self-esteem of the person because of the social stigma.

Various procedures have been employed to remove unwanted hair, including shaving, electrolysis, depilatory creams or lotions, waxing, plucking, and therapeutic anti-androgens. These conventional procedures generally have drawbacks associated with them.

U.S. patent application U.S. 2003/0180308 A1 issued to Wannemacher et al. And published on Sep. 25, 2003, “Deglycosylated ricin toxin a-chain variant” discloses ‘the primary animal models used to test ricin vaccine candidates were the rat and the mouse. The clinical signs exhibited by rats and mice after exposure to lethal quantities of aerosolized ricin are variable but include a progression of “scruffy” appearance of the hair coat, hunched posture, anorexia, conjunctivitis, and dyspnea.’

U.S. patent application 2002/0064783 A1 to Gendron et al. on May 30, 2002 and published on May 30, 2002 “Inhibition of bone tumor formation using antisense cDNA therapy” is representative of numerous patent applications relating to the use of chemical toxins, including ricin. It says, ‘unfortunately, these therapies are highly toxic to non-cancer cells and cause severe side effects, such as bone marrow suppression, hair loss and gastrointestinal disturbances.’

U.S. patent application U.S. 2002/0183248 A1 to Oldham et al And published on Dec. 5, 2002, “Method of using lectins for prevention and treatment of skin diseases and disorders” appears to have its claims directed to a method of treating disorders of dermal tissue such as hair and comprises locally administering, at a cutaneous site, at least one lectin capable of binding to the surface of pathogenic microorganisms inhabiting the hair, skin, and nails, or of binding to the superficial tissues that comprise hair, skin, and nails. Lectins that stimulate cell mitosis may also be administered to accelerate wound healing and restore the appearance of age-wrinkled skin. Lectins that coagulate blood can be administered to assist in stopping bleeding from skin lesions.

U.S. Pat. No. 5,989,267 issued to Anderson on Nov. 23, 1999, “Method of hair removal” discloses a method of hair removal comprising treating the follicle to inhibit its ability to regenerate hair (abstract). Mild toxins can be used to inactivate the hair follicle. It discloses the use of mild anti-metabolic toxins.

U.S. Pat. No. 4,720,489 issued to Shander on Jan. 19, 1988 and U.S. Pat. No. 5,096,911 issued to Ahluwalia et al. on Mar. 17, 1992 are representative of those disclosing different inhibitors to alter hair growth.

Accordingly what is needed in the art is a means to achieve effective inhibition in the growth of unwanted body hair whether androgen-sensitive,—dependant or not including almost all of the body hair in addition to the facial hair. The present invention describes these and other advantages.

### BACKGROUND OF THE INVENTION

I have discovered that removal of the hair, in preferred embodiments from its follicle e.g. by hot/cold waxing, plucking etc. or alternatively by methods like depilation or shaving, followed by the topical application; to the normal intact skin (without any nicks, burns, wounds or etc.) of the dermatologically acceptable vehicle herein described and where the active agent is incorporated, damages the follicle permanently, thereby affecting the formation of normal hair shaft.

### SUMARY OF THE INVENTION

The present invention comprises of a composition for topical application for inhibiting unwanted mammalian hair growth, particularly on facial areas, legs, arms, including armpits, torso; irrespective of the gender of the subject. The present invention also embraces a method of inhibiting mammalian hair growth by applying an effective amount of the composition to the skin after the hair is removed from the skin. The composition comprises of a hair growth-inhibiting agent and a dermatologically acceptable vehicle such as, but not limiting to, oil in water emulsion in the form of cream or lotion. Actual methods for preparing administrable compositions are also known to those of ordinary skill in the art & are described in more details in many such publications.

Other compositions like gels, soap, non-soap bars, lotions, ointments, solutions, foams, sustained release polymer films or sprays can also be used as the vehicles to carry the active ingredient to the target site. Similarly, vesicular carriers like liposomes can be used for the selective follicular delivery of the hair-growth-inhibiting agent for further accuracy in the target i.e. hair follicle. Such liposome preparations directed specifically towards hair follicle are available commercially (Invitrogen, CA).

### TECHNICAL FIELD

The hair growth-inhibiting agent that is utilised in the composition is a toxalbumin selected from a group cytotoxic proteins consisting of: abrin, ricin, & modeccin.

There is a group of cytotoxic proteins acting on eukaryotic ribosomes including those from fungi (alpha-sarcin) & higher plants (ricin, abrin &modeccin). These toxins have been known to catalytically & irreversibly inactivate 60 S ribosomal subunits affecting the activities in peptide elongation reaction. Ricin, abrin & modeccin consist of two peptide chains, A & B, linked together by a disulfide bond while alpha-sarcin is a single peptide. The B-chain binds the toxins to receptors on the cell surface, & the A-chain enters the cytoplasm & inactivates the 60 S ribosomal subunits. Irrespective of the structural differences, the mode of action of Ricin & the related lectins is known to be identical with that of alpha-sarcin in the following aspects:
1. they affect EF-1 & EF-2 associated functions of 60 S subunits &
2. they do not require energy & any cofactors.
   These lines of evidence also suggest that they also act on rRNA rather than on ribosomal proteins like aipha-sarcin.

The toxicity of abrin in mice is 75 times that of ricin (0.04 μg/kg for abrin compared to 3 μg/kg for ricin.
Ricin is a type 2 ribosome inactivating protein & a haemagglutinin. Ricin has different isoforems like Ricin D, Ricin E & RCA having minor differences in structure or the activity. Ricin is a potent cytotoxin but a weak haemagglutinin, whereas RCA (Ricinus communis agglutinin) is a weak cytotoxin and a powerful haemagglutinin. Ricin is a 66-kilodalton (kd) globular protein that makes up 1% to 5% by weight of the bean of the castor plant, Ricinus communis. The toxic heterodimer consists of a 32-kd A-chain that is disulfide-bonded to a 32-kd B-chain. The A-chain is composed of 267 amino acid residues. The toxically active A-chain of ricin is approximately 30% helical and contains 7 alpha helices. It also contains about 15% beta structure, which is made up of a five-stranded beta sheet. The B-chain is composed of 262 amino acid residues and is classed as a lectin. The toxin is stored in the matrix of the castor bean. Both chains are glycoproteins containing mannose carbohydrate groups; the two 32-kd chains must be associated for toxicity.
   Several investigators have purified and characterized ricin and have succeeded in crystallizing it. The crystal structure has been determined to 2.5 Å. The A- and B-chains are globular proteins, with the A-chain tucked into a gap between two roughly spherical domains of the B-chain. A lactose disaccharide moiety is bound to each of the spherical domains of the B-chain. The disulfide bond links amino acid 259 of the A-chain and amino acid 4 of the B-chain. Ricin is a glycoprotein, meaning that it possesses carbohydrate side chains in the form of mannose-rich N-linked oligosaccharides. In addition to binding of galatosides, these groups are important in assisting the toxin in binding to certain cell types will mannose receptors. Ricin particularly binds to mannose receptors of cells of the reticuloendothelial system. Ricin has sites with potential for binding of high mannose carbohydrate chains at asparagines 10 and 236 of the A-chain and asparagines 95 and 135 of the B-chain. The crystal structure demonstrates a putative active cleft in the A-chain, which is believed to be the site of the enzymatic action of the toxin.
Mode of Action: Ricin is a cytotoxic protein that inhibits protein synthesis by inactivating ribosomes. It has two different subunits that play distinct roles in the potent cytotoxicity of the protein. The A-subunit is an enzyme that depurinates a single base 4324 of the 28 S r-RNA, thereby inactivating protein synthesis. The B subunit, which contains two galactose-binding sites, binds cell surface glycoproteins and glycolipids containing galactose. Site 1, or the low affinity site, primarily utilizes residues Trp-37, Asp-22, Asn-46, & Gin-47 & the site 2 utilizes Tyr-248, Asp-234, Gln-256, & Asp-255 for galactose recognition.

The mechanism of inactivation is the hydrolysis of a N-glycosidic bond adjacent to the alpha-sarcin site in 28 S r-RNA. The specificity of the effect of ricin A-chain, the catalytic subunit of the toxin, on ribosomes is remarkable. Only one glycosidic bond in 28 S rRNA is cleaved, & the other rRNAs are unaffected. Ricin A-chain cleaves the N-glycosidic bond in 28 S rRNA at A-4324; that nucleotide occurs in a sequence, AGUACGAGAGGMC, & is highly conserved.

Effect of pH:
It has earlier been shown that the ability of abrin & ricin to inhibit protein synthesis increases with increasing pH up to 8.5. In contrast, when pH reduces below neutrality, the cells become increasingly insensitive & at pH 6.0 the toxins are unable to inhibit protein synthesis (11). The low pH in the medium does not facilitate entry of plant toxins. It has been found out that ricin binds rapidly to cell surface & that the total amount of abrin & ricin bound to cells does not vary much between pH 6.0& 8.0. It is well established that at 37° C. extensive endocytosis of the toxins takes place (10). The conformational properties of RTA are ideally suited to its translocation from the ER, in that the conformational labiality and non-cooperatively of the protein at pH 7.0 permits relatively frequent unfolding events and population of partially unfolded molecules even close to ambient temperature (13). At neutral pH, the ricin molecule is very compact. The most important & interesting effect exerted by pH on the ricin molecule is the one that occurred at pH below 7 on both its conformation & affinity for galactosides. When the pH is lowered from neutrality, the affinity for ricin for galactosides decreases concomitantly with the changes in the molecule conformation; subsequently at lower pH, this decrease is accompanied by an alteration in binding capacity (14).
The composition comprising of the active agent of the of the present invention will typically be adjusted to a pH in the range of 6.0 to 8.5, preferably close to neutrality & optionally comprising of a pH buffer.

Dermal Toxicity:

Toxicity of ricin also varies with route of challenge. In laboratory mice, the approximate dose that is lethal to 50% of the exposed population (LD₅₀) and time to death are, respectively, 3 to 5 micrograms/kg and 60 hours by inhalation, 5 micrograms/kg and 90 hours by intravenous injection, 22 micrograms/kg and 100 hours by intraperitoneal injection, 24 micrograms/kg and 100 hours by subcutaneous injection, and 20 mg/kg and 85 hours by intragastric administration. Low oral toxicity reflects poor absorption of the toxin from the gastrointestinal tract. Higher toxicities by other routes may be directly related to accessibility of target-cell populations and the ubiquity of toxin receptors throughout the cells of the body. When skin tests were performed on mice, no dermal toxicity was observed at the 50-micrograms spot (5).

LD 50 Dose in lab mice (mcg/kg body weight)

| Route of Challenge | Ricin | Abrin |
|---|---|---|
| Subcutaneous | 24 | |
| Gastrointestinal | 30 | |
| Parenteral | 3-5 | 0.04 |
| Aerosol | 3-5 | 0.04 |

Dermal exposure is unable to achieve toxicity. Toxicity of ricin also varies with route of challenge. To be absorbed dermally, ricin must be enhanced with a strong solvent such as DMSO.
Purification of proteins: Ricin & Abrin are readily available from commercial sources (Vector Labs, Sigma Chemical co.). Methods of isolating Ricin & Abrin are also well known to those of ordinary skill in the art.
Before administration of the vehicle containing the active ingredient the hair is removed in a preferred embodiment from the follicle e.g. by waxing/plucking or alternatively by other methods e.g. by shaving. Methods of hair removal, which pluck the hair from the follicle, are known to initiate the anagen phase, & also it exposes the hair follicle assembly responsible for synthesis of hair shaft to the active agent.
The topical compositions are the formulations containing therapeutically effective amounts of the respective active agent and pharmaceutically acceptable auxiliary substances as required to approximate physiological conditions such as but not limiting to pH adjusting and buffering agents, toxicity adjusting agents and the like, vesicular carriers like liposomes for carrying the active agent to the hair follicle, along with the usual components of the particular carrier like stearic acid and its derivatives, cetyl alcohol and its derivatives, mineral oils, citric acid, preservatives, colours, fragrance etc. as described in different pharmaceutical publications.
Animal study—Animal study using albino mice showed permanent reduction in the number of follicles in all mice. The biopsies of the animals showed no adverse reaction on the skin of the animals. The treated patches show number of empty follicles. As the active agent is a cytotoxic agent, the damage is permanent resulting in “Empty Follicles”.

Applications:
1. For the treatment of hirsuitism & hypertrichosis for androgen-dependant/-independent hair re-growth after removal from face (including beard areas in men), ear, torso, back, pubic area, underarm, arms & legs (including digits)
2. For cosmetic purpose in humans, irrespective of gender.
3. For cosmetic treatment of other mammals like show-animals, for example dogs, horses.
4. For pharmaceutical purpose.
   At the end of the description of the invention, it should be understood that without departing from the spirit of the invention or the essential characteristics, it might be embodied in other variations. The embodiments described here should be considered as illustrative but not restrictive. All variations & forms, which come within the meaning & range of equivalency of the claim, are intended to be embraced therein.

### REFERENCES

1. Lehninger A. L. Principles of Biochemistry: Chapter 29, Chapter 3
2. J M Lord, L M Roberts and J D Robertus. Ricin: structure, mode of action, and some current applications (1994). The FASEB Journal, Vol 8, 201-208
3. Yaeta E., Kunio T. The RNA N-Glycosidase activity of Ricin A-chain. The Journal Of Biological chemistry (1988) 263: 8735-8739.
4. Yaeta Endo, Kazuhiro Mitsui, Mitsuyoshi Motizuki & Kunio Tsurugi. The Mechanism of action of Ricin & Related Toxic Lectins on Eukaryotic Ribosomes (1987). The Journal of Biological Chemistry (1987). Vol 262(12), 5908-5912.
5. David R. Franz, Nancy K. J. Ricin Toxin. Textbook of Military Medicine: Medical Aspects of Chemical and Biological Warfare: Chapter 32.
6. David R. Franz, Understanding the Threat. Virtual Naval Hospital-Defense Against Toxin Weapons: Chapter 1.
7. Sarah L. Corbett Biology Senior Seminar-Valdosta State University. Terror, Murder, and Medicine: the biological effects and abuses of ricin, a plant lectin isolated from Ricinus communis.
8. Stenn K. S., Paus R. 2001 Controls of Hair Follicle Cycling. Physiological Review 81: 449-494,
9. Newton Dianne I., Wales Richard, Richardson Peter T., Walbridge S., Saxena Shailendra K., Ackerman Eric J., Roberts Lynn M., Lords Michael J., Youle Richard J. Cell surface and intracellular functions for Ricin galactose binding. The Journal OF Biological chemistry 1992; 267:11917-11922.
10. Sandvig K. & Olsnes S. Entry of toxic proteins abrin, modeccin, ricin & diphtheria toxin into cells-Requirement for Calcium. 1982. J Biological Chemistry; 257(13) 7495-7503.
11. Sandvig K. & Olsnes S. Entry of toxic proteins abrin, modeccin, ricin & diphtheria toxin into cells-Effect of pH, metabolic inhibitors& ionophores & evidence of toxin penetration from endocytotic vesicles. 1982. J Biological Chemistry; 257(13) 7504-7513.
12. Bushueva T L, Tonevitskii A G. Effect of pH on the conformation & stability of the plant toxin ricin. Mol Biol (Mosk). (1987) 21(2): 41421.
13. Argent R. H. et al. Ribosome-mediated Folding of Partially Unfolded Ricin A-chain. J Biol Chem vol. 257 (13) 9263-9269.
14. Frenoy J P. Effect of physical environment on the conformation of ricin. 1986. Influence of low pH. Biochem J. 1986; 240(1): 221-226.
15. S. Gupta, A. Domashenko, G. Cotsarelis. The hair follicle as a target for gene therapy. European Journal of Dermatology vol 11, number 4, 353-6 2001
16. Domashenko A, Gupta S, Cotsarelis G. Efficient delivery of transgenes to human hair follicle progenitor cells using topical lipoplex. Nat Biotechnol. 2000 April; 18(4): 420-3.
17. Wilkinson J. B., Moore R. J. Harry's Cosmetology. Chemical Publishing.

1. A topical composition for inhibiting mammalian hair growth, which comprises of a hair-growth-inhibiting active agent, which is a toxalbumin selected from a group of cytotoxic lectins such as ricin, abrin, and modeccin, dispersed in a dermatologically acceptable vehicle, such as herein described.

2. The composition of claim 1, wherein said active agent is Ricin.

3. The composition of claim 1, wherein said active agent is Abrin.

4. A method of inhibiting mammalian hair growth, which comprises of applying, the said topical composition, at a level sufficient to provide up to 20 milligrams of active agent per square centimeters of normal, intact skin; after the hair is removed, preferably from the follicle, for example by, but not limiting to, waxing, plucking, epilating or alternatively by shaving or depilation; according to claim 1.

5. The method of claim 4, wherein the said topical composition is applied to normal intact human skin.
